(19) United States
(12) Patent Application Publication
(10) Pub. No.: US 2017/0242023 A1
(43) Pub. Date: Aug. 24, 2017

(54) Methods Of Detecting Ectopic Pregnancy

(71) Applicant: Map IP Holding Limited, Cambridgeshire (GB)

(72) Inventors: Raymond Kruse Iles, Cambridgeshire (GB); Stephen Andrew Butler, Cambridgeshire (GB)

(73) Assignee: Map IP Holding Limited, Cambridgeshire (GB)

(21) Appl. No.: 15/323,409

(22) PCT Filed: Jul. 2, 2014
(86) PCT No.: PCT/GB14/52009

§ 371 Date: Dec. 31, 2016

(51) Int. Cl. G01N 33/68; G01N 33/76; G01N 33/74

(52) U.S. Cl. CPC G01N 33/689; G01N 33/743; G01N 33/76; G01N 2800/368; G01N 2333/575; G01N 2333/59; G01N 2333/4725; G01N 2800/50

(57) ABSTRACT

A method for determining the likelihood of an ectopic pregnancy and spontaneous miscarriage is described, by measuring the levels of markers, especially hCG and CA-125 which have been found to be characteristic of these conditions. Preferably measuring these biomarker levels at earliest possible presentation of patients with general clinical symptoms and applying cut-off values described determines the likelihood of an ectopic pregnancy and spontaneous miscarriage.

### FIELD OF THE INVENTION

The present invention relates to a method for determining the likelihood of an ectopic pregnancy and spontaneous miscarriage by measuring the levels of markers which have been found to be characteristic of these conditions.

### BACKGROUND TO THE INVENTION

Ectopic pregnancy is the leading cause of maternal mortality in the first trimester in both the United Kingdom and North America where death is secondary to tubal or interstitial rupture. Although the cause is maternal circulatory collapse, delayed intervention and misdiagnosis is the clinical cause. Thus, early accurate diagnosis prior to rupture is vital.

Currently, diagnosis of ectopic pregnancy is based on transvaginal ultrasound and quantitative serum measurement of human chorionic gonadotropin (hCG). Increasingly sensitive ultrasound and hCG assays are effective for diagnosis of ectopic pregnancy in most women, and have reduced the need for the more invasive diagnostic laparoscopy. However, diagnostic problems still exist in these established methods and in 40-50% of cases the diagnoses made at presentation are incorrect. An ectopic pregnancy is likely if transvaginal ultrasound shows an empty uterus and a gestational sac in the adnexal region; however, visualisation of the gestational sac is size and thus age-dependent, requiring a minimum size of 5-6 mm. Therefore, very early ectopic pregnancies are missed. The hCG discriminatory zone is the threshold level of serum hCG, currently 1000-1500 IU/mL, at which point a gestational sac is expected to be reliably visualized on ultrasound. Above this threshold, a transvaginal ultrasound finding of an empty uterus with presence of an adnexal mass is strongly suggestive of ectopic pregnancy. However, the results may be inconclusive, requiring repeat follow up over days during which there is a prolonged risk of tubal rupture. Serial measurements of serum hCG becomes the mainstay of diagnosis when transvaginal ultrasound is inconclusive, and is based on the distinguishing doubling rates of serum hCG over 48 h. The discriminatory boundaries of this method appear to be ambiguous with different doubling rates for ectopic pregnancy described at <66% increase over 48 h, and more recently at <53% increase over 48 h. Furthermore, 13% of ectopic pregnancies do show an unexpected increase beyond the cutoff rate of 66%—particularly in very early ectopic pregnancies. Patient anxiety during delayed diagnosis is yet another shortcoming to current diagnostic protocols. These problems collectively pose a research need for a single-point diagnosis of ectopic pregnancy at initial presentation.

Human chorionic gonadotrophin (hCG) is a heterodimeric glycoprotein hormone composed of an alpha- (hCGα) and a beta subunit (hCGβ) held together non-covalently. Total hCG (hCGt) consists of hCG variants which include intact hCG, the free beta subunit (hCGβ), and the hyperglycosylated isoform (hCGh). Previously, it has been found that urine hCG variants had less significant performance compared to those of serum in distinguishing ectopic pregnancy from viable pregnancy. Single measurements of serum hCGt and hCG produced promising results in distinguishing ectopic pregnancy from viable pregnancy. Serum hCGβ at 281 pmol/L cutoff had 100% sensitivity and 79% specificity; and serum hCGt at 1053 pmol/L cutoff had 100% sensitivity and 68% specificity. hCGh was not included as no assay was generally available. It was also found that both hCGt and hCG could not distinguish ectopic pregnancy from spontaneous miscarriage.

Progesterone is a steroid which is produced by the hCG-maintained corpus luteum during pregnancy. Serum progesterone has been shown to be lower in ectopic pregnancy compared to viable pregnancy but differing cutoffs have been suggested ranging from 5 to 22 ng/mL.

CA125 is a protein produced by epithelia of several tissues including the peritoneum, and fallopian tubes. Though elevated serum levels are found in ovarian cancer, they are primarily a feature of peritoneal irritation or perforation which may occur during ectopic pregnancy. Significantly low-, and high levels, as well as nondiscriminatory results have been found in ectopic pregnancy compared to intrauterine pregnancy.

The present application provides a method for determining the likelihood of an ectopic pregnancy or spontaneous miscarriage. The present invention describes a method and an algorithm which can detect ectopic pregnancy at initial presentation and circumvent delays from current protocols. In addition the methods of the invention can distinguish between ectopic pregnancy, viable pregnancy, and spontaneous miscarriage during early pregnancy.

Human chorionic gonadotropin (hCG) is a glycopeptide hormone produced by the syncytiotrophoblasts of the fetal placenta, and has a molecular weight of about 38 kilodaltons. It can be detected by immunoassay in the maternal urine within days after fertilization. The intact hCG molecule is a heterodimer comprising a specific β subunit non-covalently bound to an α subunit, which is common to other glycoproteins. It is a hetrodimeric glycoprotein hormone with 8 glycosylation sites containing four N-linked oligosaccharides and four O-linked oligosaccharides. The N-linked oligosaccharides are attached to the polypeptide chain by β-N-glycosidic bonds on asparagine residues; two are on the α and two are on the β-subunit. They share the same basic structural characteristics: N-acetyl glucosamine (GLcNAc) is attached to an asparagine residue followed by another GLcNAc, mannose and two more branches of mannose. This is the monantennary pentasaccharide core with the remaining components being variable. The O-linked oligosaccharides are attached by α-O-glycosidic bonds onto serine residues of the β-subunit carboxyl terminal peptide.

Carbohydrate heterogeneity has been extensively reported for the free β-subunit of hCG (hCG β) with variable mono-, bi- and triantennary carbohydrate structures being found in normal and abnormal pregnancies.

The degradation product of the β-subunit of hCG known as β-core fragment (hCG β cf) is composed of peptides from the β-subunit of hCG, i.e peptides β-40 and β 55-92, connected by four disulfide bridges. It retains many of the antigenic determinates of the original hCG β molecule prior to metabolism, which occurs primarily in the kidney. The β 6-40 polypeptide chain contains the two hCG β N-linked carbohydrate moieties, although the oligosaccharides are truncated due to metabolism.

CA-125 sometimes called cancer antigen 125, carcinoma antigen 125, or carbohydrate antigen 125; is also known as mucin 16 or MUC16. It is a protein that in humans is encoded by the MUC16 gene. MUC16 is a member of the mucin family glycoproteins. CA-125 has found application as a biomarker that may be elevated in the blood of some patients with specific types of cancers, or pregnancy and other benign conditions. CA-125/Mucin 16 is a membrane associated mucin that possesses a single transmembrane domain. A unique property of MUC16 is its large size. MUC16 is more than twice as long as MUC1 and MUC4 and contains about 22,000 amino acids, making it the largest membrane associated mucin.

MUC16 is composed of three different domains:

An N-terminal domain

A tandem repeat domain

A C-terminal domain

The N-terminal and tandem repeat domains are both entirely extracellular and highly and variably O-glycosylated. All mucins contain a tandem repeat domain that has repeating amino acid sequences high in serine, threonine and proline. The C-terminal domain contains multiple extracellular SEA (Sea urchin sperm protein, Enterokinase, and Agrin) modules a transmembrane domain, and a cytoplasmic tail. The extracellular region of MUC16 can be released from the cell surface by undergoing proteolytic cleavage. CA-125/MUC16 is thought to be cleaved at a site in the SEA modules.

Progesterone or pregn-4-ene-3,20-dione, often abbreviated to P4, is a C-21 steroid hormone involved in the female menstrual cycle, pregnancy (supporting gestation by maintaining the endometrium as the hormone that prevents endometrial cell apoptosis) and embryogenesis of humans and other species. Progesterone belongs to a class of steroids called progestogens, and is the major naturally occurring human progestogen.

Progesterone is produced in the ovaries (by the corpus luteum), the adrenal glands and, during later pregnancy, in the placenta. Increasing amounts of progesterone are produced during pregnancy. At first, the source is the corpus luteum that has been “rescued” by hCG from the conceptus. However, after the 8th week, production of progesterone shifts to the placenta. The placenta utilizes maternal cholesterol as the initial substrate, and most of the produced progesterone enters the maternal circulation, but some is picked up by the fetal circulation and used as substrate for fetal corticosteroids. At term the placenta produces about 250 mg progesterone per day.

### Statements of the Invention

In one aspect the invention provides a method of determining the likelihood of an ectopic pregnancy or spontaneous miscarriage comprising measuring the level of one or more markers selected from total hCG(hCGt); free beta subunit of hCG (hCG(3), hyperglycosylated form of hCG (hCGh), CA125 and progesterone in a sample obtained from a pregnant woman. Preferably the level of hCGt or CA125 is measured, more preferably the levels of hCGt and CA125 are measured. Alternatively the level of hCGh is measured.

The method may further comprise analysing the results obtained from an ultrasound scan to determine the presence of a foetal sac and optionally a foetal heartbeat.

### DETAILED DESCRIPTION

In one aspect the invention provides a method of determining the likelihood of an ectopic pregnancy or spontaneous miscarriage comprising measuring the level of one or more markers selected from total hCG(hCGt); free beta subunit of hCG (hCG(3), hyperglycosylated form of hCG (hCGh), CA125 and progesterone in a sample obtained from a pregnant woman.

As used herein “determining the likelihood” includes detecting an ectopic pregnancy as well as providing a level of risk of an ectopic pregnancy or spontaneous miscarriage.

The levels of individual markers can be measured, or the combination of two or markers can be measured. For example combinations of markers such as

hCGT, hCGf3 and CA125 (TBC);

hCGT and hCGβ (TB);

hCGt and CA125 (TC);

hCGT, hCGh and hCGβ (THB) or

hCGT, hCGh, hCGβ, progesterone and CA125 (THBPC) can be measured.

The ratio of hCGt to hCGh (T/H) can also be used.

The level of the markers, which are present as proteins or polypeptides in the sample, can be measured using antibodies or immunoassay techniques. Suitable methods are known to the skilled person and may include ELISAs, Radioimmunoassays, surface plasmon resonanace, and include both competitve and non-competitve assays.

The method of the invention can be used to distinguish between a normal viable pregnancy and an ectopic pregnancy. All of the markers have been found to be present at significantly lower levels in ectopic pregnancies compared to a viable pregnancy. The best single marker for determining the likelihood of, or detecting an ectopic pregnancy is hCGt (T), but the best results are obtained using a combination of hCGt (T) and CA125(C). Thus, preferably the level of hCGt is measured, more preferably the levels of hCGt and CA125 (TC) are measured. A level of ≦3736mIU/ml hCGt or an Area under the curve (AUC) of ≦0.878 can be used to detect an ectopic pregnancy. A level of ≦41.98 U/ml CA125 can also be used to detect an ectopic pregnancy. The level of CA125 can be measured if the level of hCGt is indicative of an ectopic pregnancy and vice versa. In addition a level of ≦80,893.5 or AUC≦0.912 can be used as a combined level for hCGt×CA125 (TC) to detect an ectopic pregnancy. Alternatively the level of hCGh is measured. Preferably the marker is not progesterone.

The number of false positives for detecting an ectopic pregnancy can be reduced by combining the results obtained from measuring the levels of the markers with the results of an ultrasound scan. Thus the method of the invention may further comprising analysing the results obtained from an ultrasound scan carried out to determine the presence of a foetal sac and optionally a foetal heartbeat.

The method of the invention can be used to distinguish between an ectopic pregnancy and a pregnant woman likely to suffer a spontaneous miscarriage. The levels of a marker, or a combination of markers selected from C, TC, TBC, T, TB, T/H, P or B can be used to distinguish between an ectopic pregnancy and a pregnant woman likely to suffer a spontaneous miscarriage. A level of ≦42.029 U/ml CA125 can also be used to distinguish between an ectopic pregnancy and a pregnant woman likely to suffer a spontaneous miscarriage. In addition a level of ≦43276.8 can be used as a combined level for hCGt x CA125 (TC) used to distinguish between an ectopic pregnancy and a pregnant woman likely to suffer a spontaneous miscarriage.

The method of the invention can be used to distinguish between a normal viable pregnancy and a pregnant woman likely to suffer a spontaneous miscarriage. The levels of a marker, or a combination of markers selected from T, TC, TBC, P, THBPC, THB, H or B can be used to distinguish between a normal viable pregnancy and a pregnant woman likely to suffer a spontaneous miscarriage.

The method of the invention is carried out on a sample obtained up to, and including the second trimester of pregnancy. Preferably, the maternal sample is from a pregnant woman at between 4 and 16 weeks gestation, for example 4, 5, 6, 7, 8, 9, 10, 11, 12, 13, 14, 15 or 16 weeks gestation. More preferably the maternal urine sample is from a pregnant woman at between 5 and 8 weeks gestation.

The sample may be any biological fluid sample such as blood, serum, saliva, and urine. Preferably, the sample is a serum sample. The sample may be diluted or processed (concentrated, filtered, etc.).

Also described is a method of determining the likelihood of an ectopic pregnancy or spontaneous miscarriage comprising

a) obtaining a sample from a pregnant woman;

b) measuring the level of one or more markers selected from total hCG(hCGt); free beta subunit of hCG (hCG(3), hyperglycosylated form of hCG (hCGh), CA125 and progesterone;

c) Determining the likelihood of an ectopic pregnancy or spontaneous miscarriage by comparing the level of a marker measured with a cutoff value, wherein a reading below the cutoff value is indicative of an ectopic pregnancy or spontaneous miscarriage.

Preferably the method comprises comparing the levels of the marker(s) with those obtained in a sample from a woman with a normal viable intrauterine pregnancy to determine whether said levels from said sample from a pregnant woman are indicative of an ectopic pregnancy or an increased risk of a spontaneous miscarriage. The method may further comprise carrying out an ultrasound scan to detect a gestational sac and optionally a fetal heartbeat.

In this specification, the verb “comprise” has its normal dictionary meaning, to denote non- exclusive inclusion. That is, use of the word “comprise” (or any of its derivatives) to include one feature or more, does not exclude the possibility of also including further features. The word “preferable” (or any of its derivates) indicates one feature or more that is preferred but not essential.

All or any of the features disclosed in this specification (including any accompanying claims, abstract and drawings), and/or all or any of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

The application will now be described in the examples below which refer to the following figures:

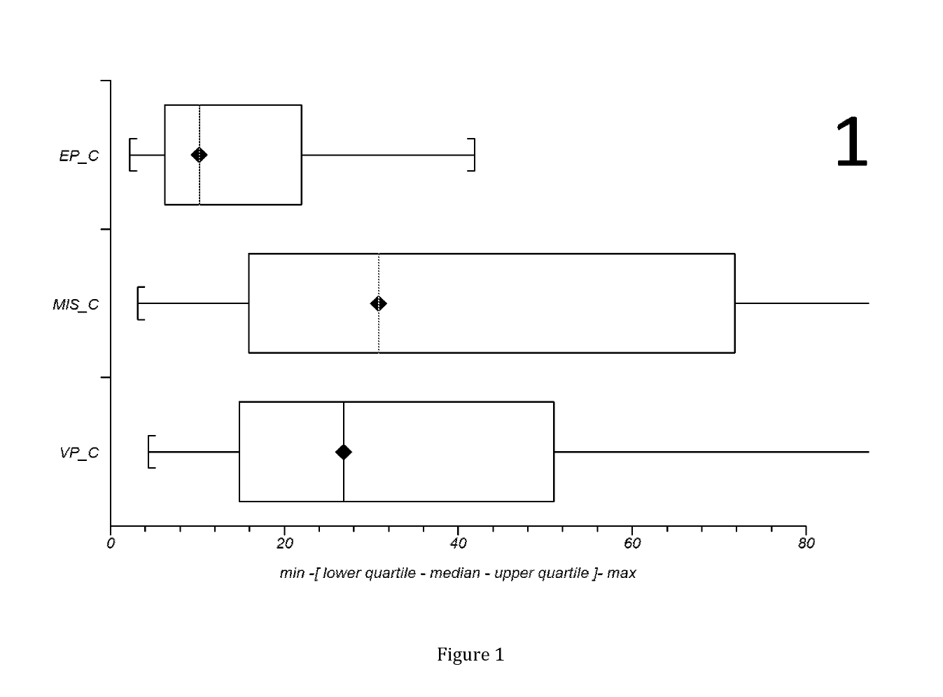

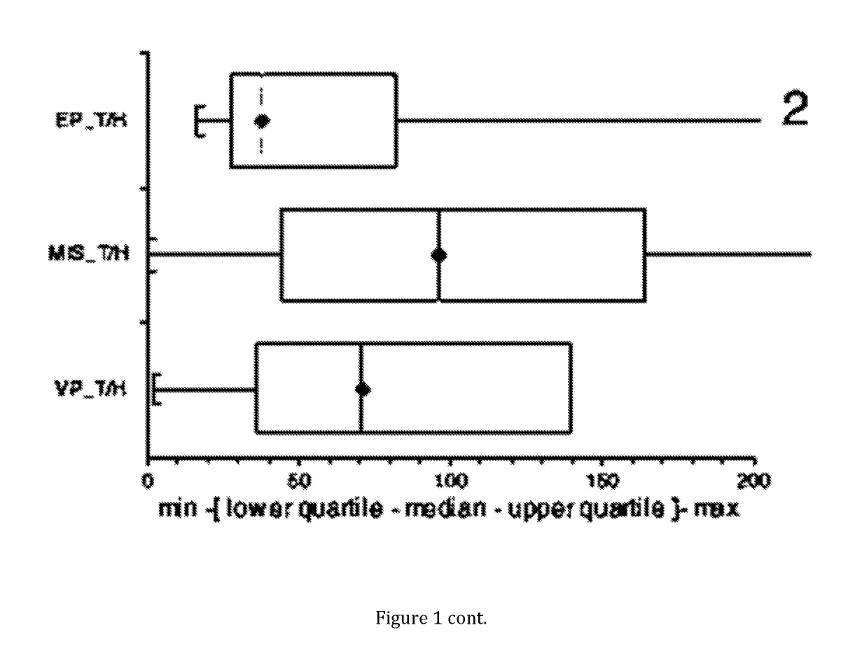

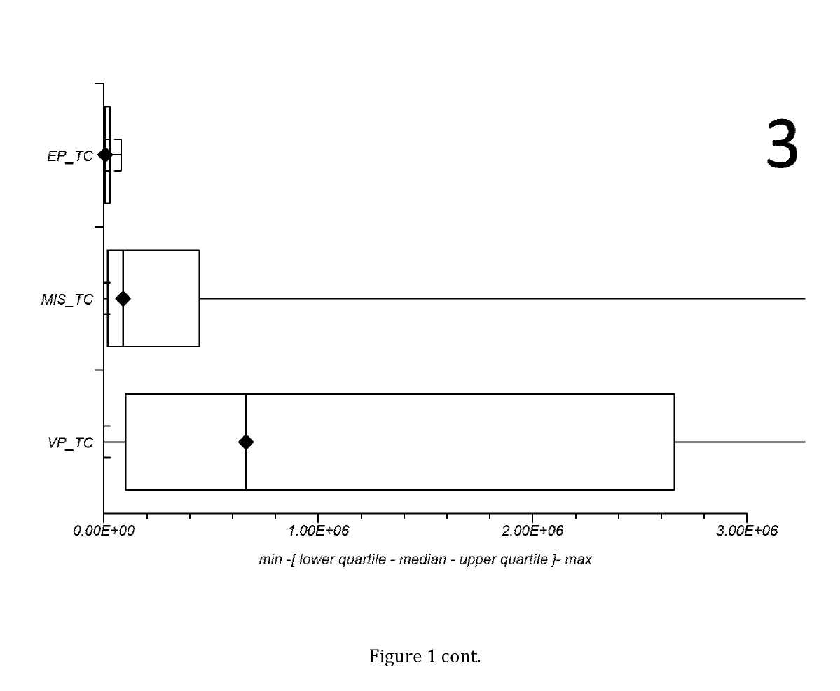

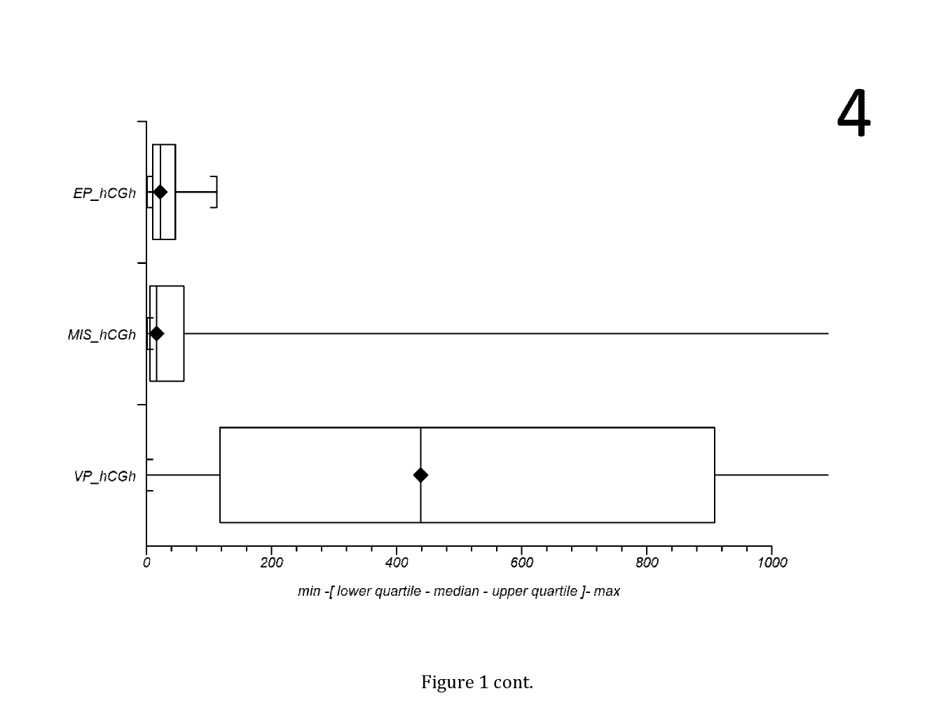

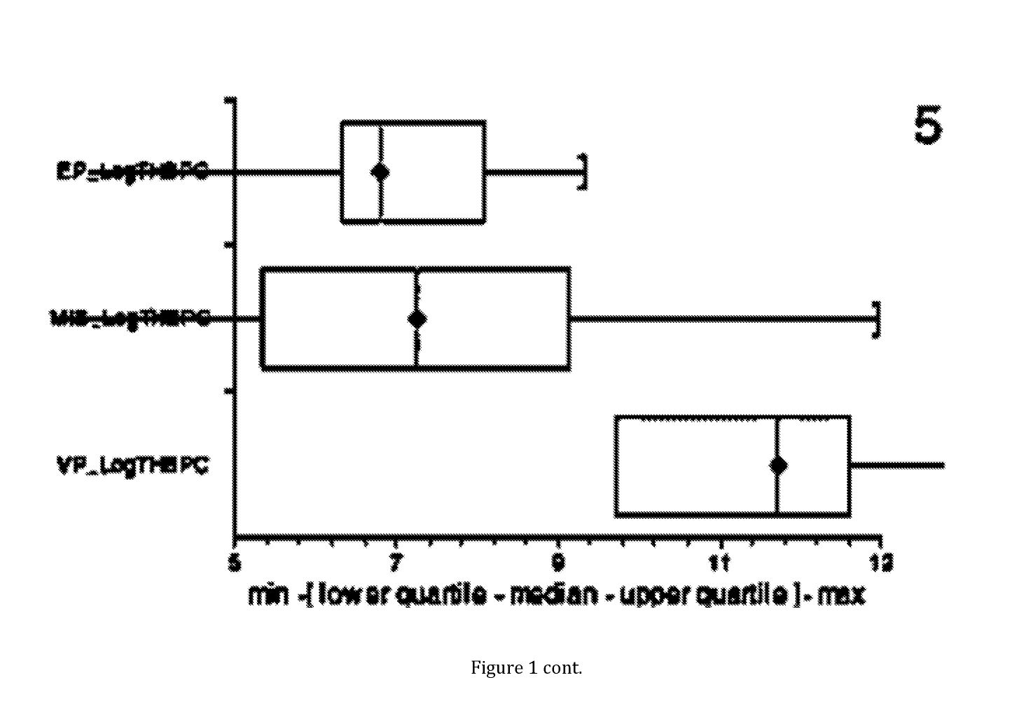

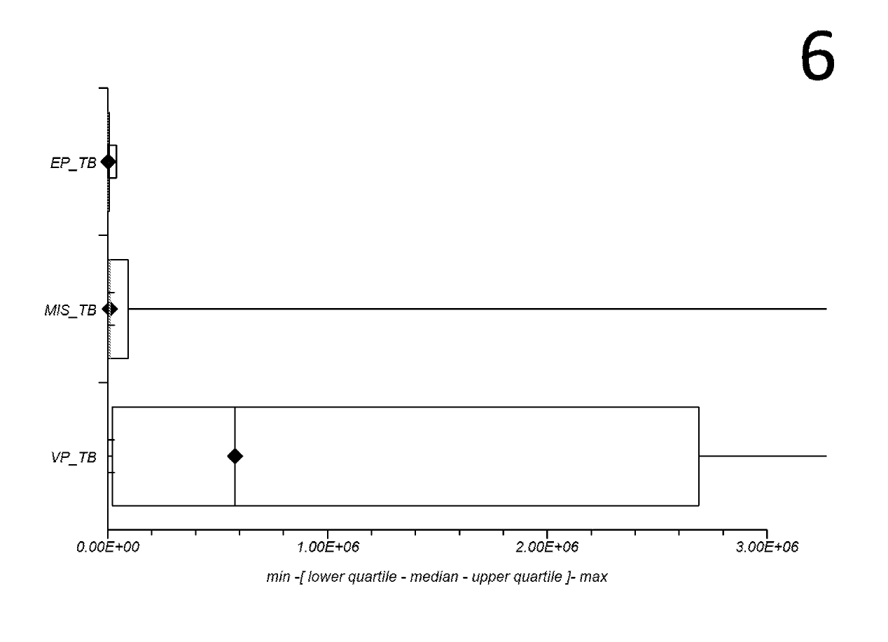

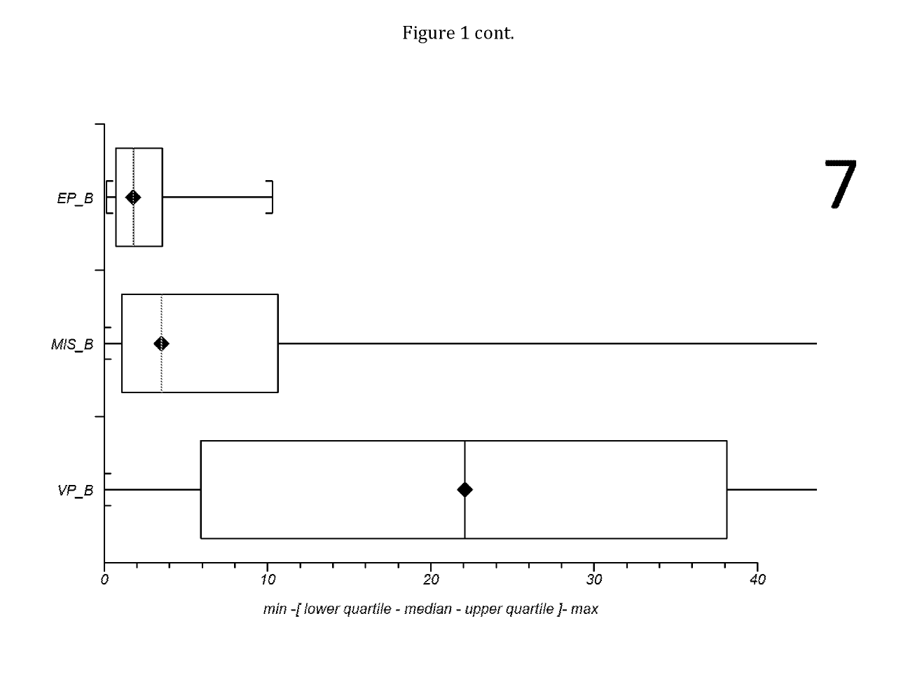

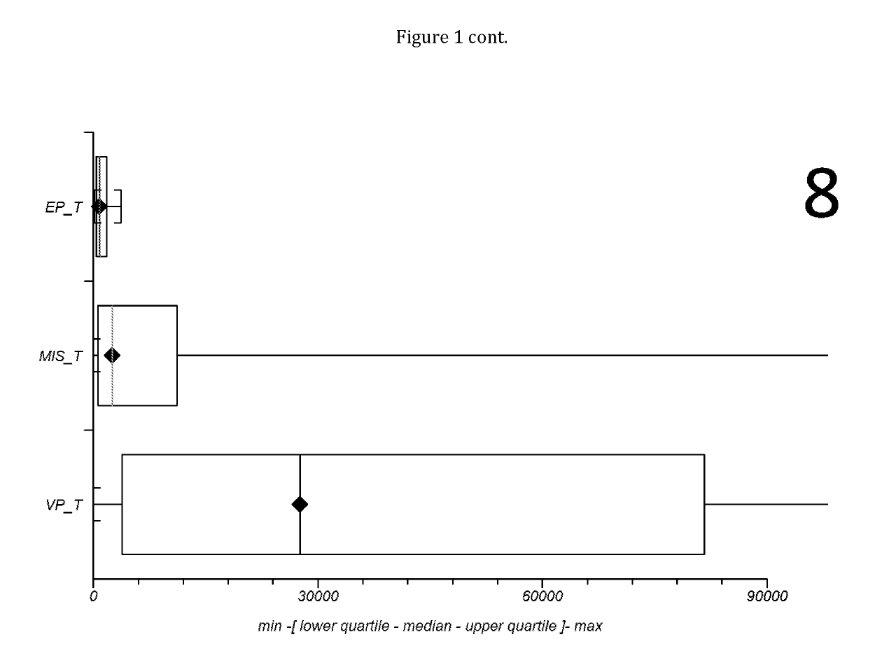

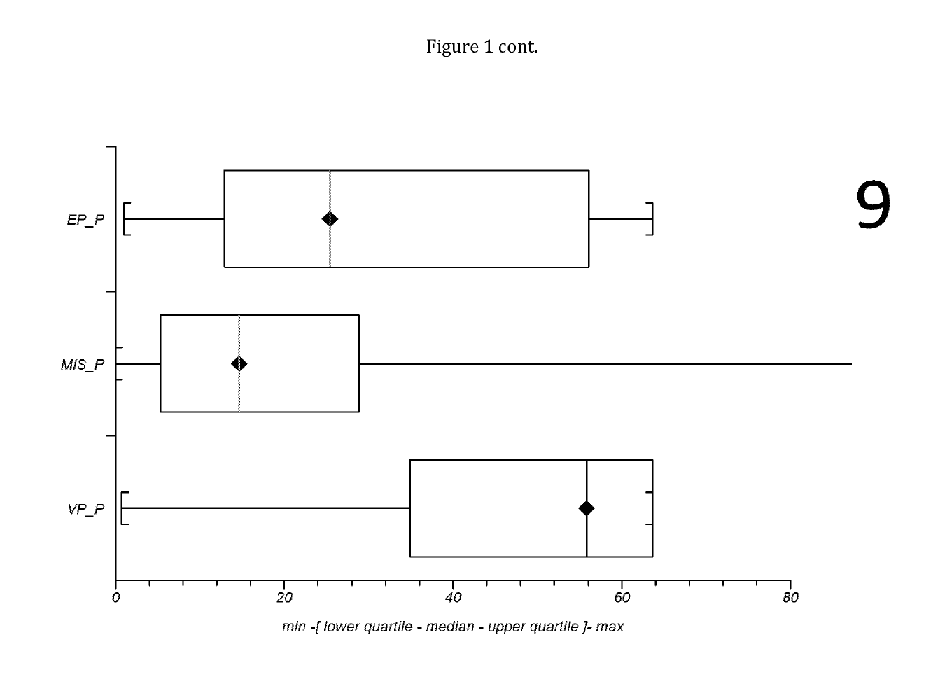

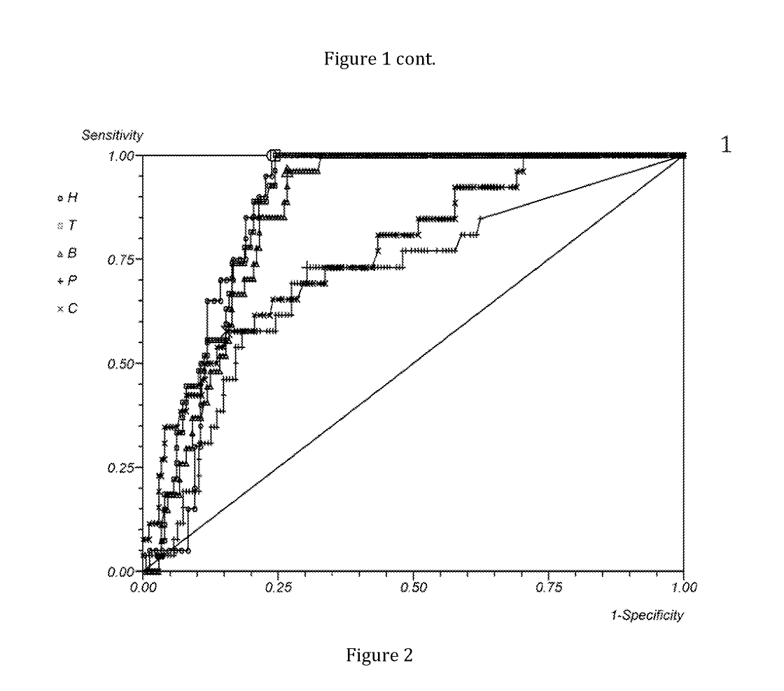

FIG. 1 shows Box plots of hCGt, hCG(3, hCGh, progesterone and CA125 in the sera of women with viable pregnancy, spontaneous miscarriage, and ectopic pregnancy at presentation. 1=CA125; 2=TH ratio; 3=TC; 4=hCGh; 5=THBPC; 6=TB; 7=hCGb; 8=hCGt; 9=progesterone. T=hCGt; B=hCGβ; H=hCGh; C=CA125; P=progesterone.

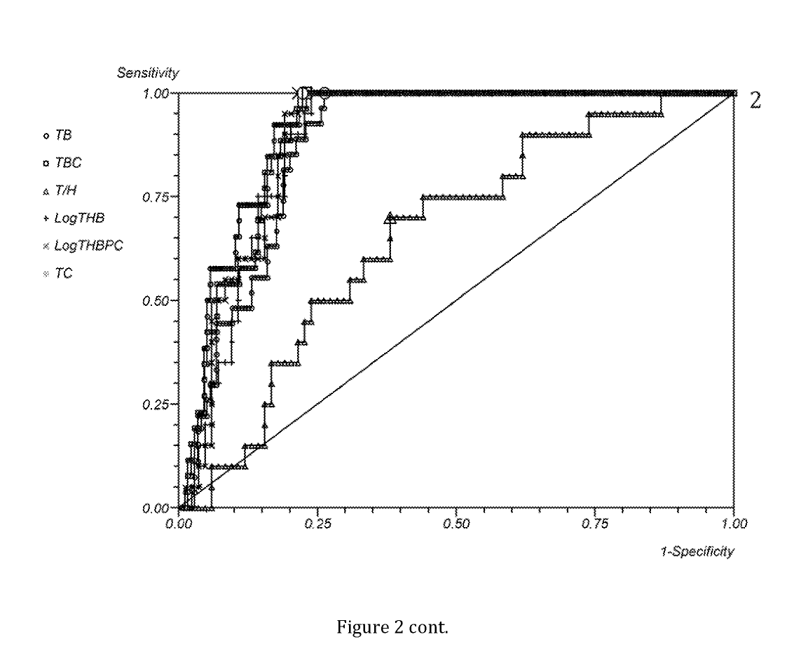

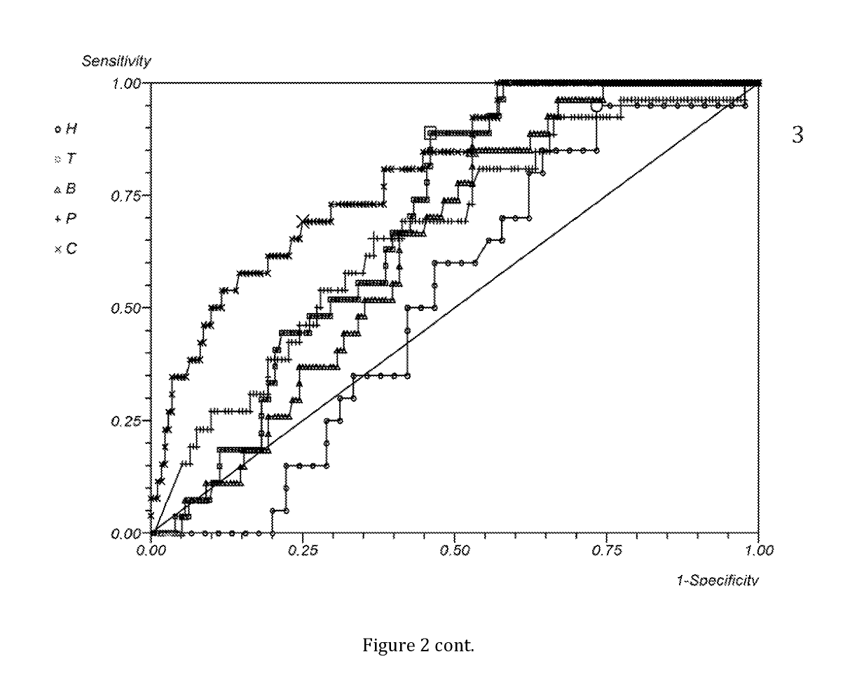

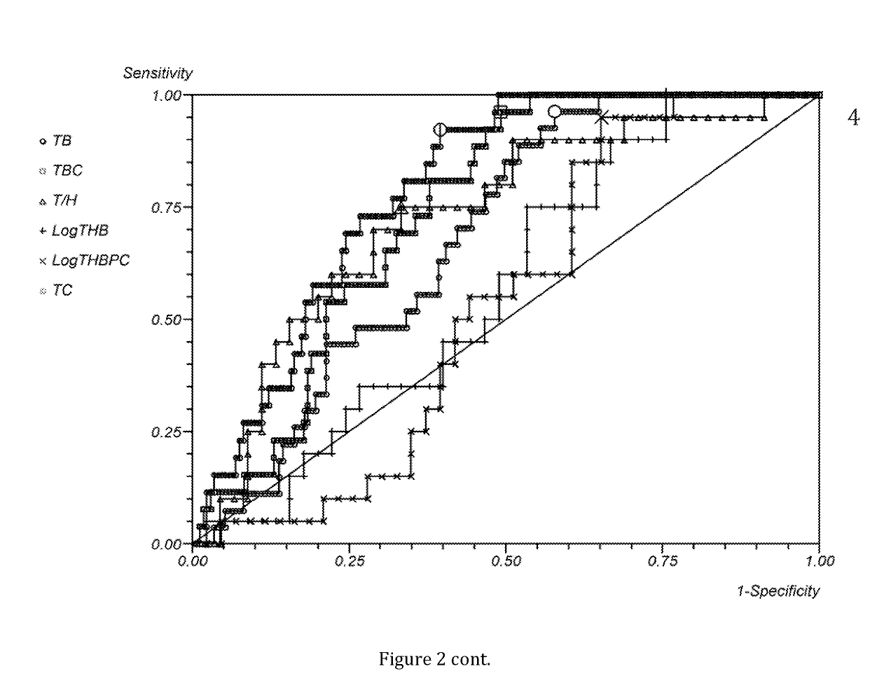

FIG. 2 shows ROC analyses for (top) inclusion of ectopic pregnancy and exclusion of viable pregnancy; and (bottom) inclusion of ectopic pregnancy and exclusion of spontaneous miscarriage for hCGt, hCGβ, hCGh, progesterone and CA125. Single- and combined markers are in 1, 3 and 2, 4, respectively. T=hCGt; B=hCGβ; H=hCGh; C=CA125; P=progesterone.

### EXAMPLE 1

### Materials and Methods

### Women and Samples

Serum samples were collected between September 2002 and March 2003 at the accident and emergency department (AE) and early pregnancy assessment unit (EPAU) of Homerton NHS Hospital Trust London, from 441 women at initial presentation with a positive pregnancy test in the first trimester, lower abdominal pain and/or vaginal bleeding. Gestational age was estimated based on date of last menstrual period as is standard practice in EPAU. The final diagnoses were confirmed through follow up on pregnancy outcome, laparoscopy, and histology of products of conception. 7/441 patients were lost to follow up and 58/441 samples were incorrectly stored and therefore disposed of The remaining 376 patients had a final diagnoses of viable pregnancy in 175 women; spontaneous miscarriage (inevitable, incomplete, complete) in 175 women; and ectopic pregnancy in 26 women. 2 patients initially diagnosed as ectopic pregnancies by current assessment practice were confirmed as spontaneous miscarriage upon follow up. Clinical data for the initial and final diagnoses were obtained by blinded personnel and matched for each patient. Samples were stored at −20 ° C. Ethical approval was obtained from the North East Thames Region Health Authority Ethics Committee.

Hormone Assays

Sample analyses were carried out by automated analysis on the DPC IMMULITE 2000 platform in accordance with the manufacturer's instructions. Sera of 376 samples were assayed for matched hCGt, hCGβ (referring to the free beta subunit of hCG and not a result of an hCG assay designated as ‘βhCG’ but measuring both free beta subunit and intact hormone hCG (total hCG)), progesterone, and CA125. Sufficient residual sera was available from 149 patient samples for hCGh measurement using the ITA (Invasive Trophoblast Antigen—hCGh) assay performed using the discontinued Nichols Institute Diagnostics Advantage system. Quality controls for the lower-, mid- and upper linear range were run concurrently with test samples. The detection limits were hCGt, 0.4 mIU/mL; hCGO, 0.02 ng/mL; progesterone, 0.2 ng/mL; and CA125, 1 U/mL.

Data were recorded in Excel 2007 and statistical analyses were performed with Stats-Direct software. Differences between median concentrations were analysed using Kruskal-Wallis (Conover-Inman) tests for single- and combination markers. The assigned value for significance was P<0.05, and optimum cutoff during ROC analyses was based on equal sensitivity: specificity weighting except for CA125 during ectopic pregnancy against spontaneous miscarriage where cutoff was optimised for 100% sensitivity.

Ultrasound Detection of Intrauterine Pregnancy

As a matter of routine the EPAU report in patient's notes whether a foetal sac and foetal heart beat are detected upon ultrasound examination of patients referred to the on call ultra-sonographer. Thus we could retrospectively compare biochemical analysis with an ultrasound finding. In subsequent analysis patient notes were examined to see if ultrasound findings would reduce false positive arising from any biochemical screening due to spontaneous miscarriage.

Results

The gestational ages were similar for viable pregnancy, spontaneous miscarriage, and ectopic pregnancy with a mean of 7.3, 7.6, and 6.3 weeks, respectively (Table 1). This eliminated bias associated with variability of marker concentration with gestational age.

The current ultrasound based screening for ectopic pregnancy detected 11 (42%) of the ectopic pregnancy at presentation and 12 (46%) on follow-up scan, but 2 (8%) were only correctly identified upon emergency re-admission, both women presented with hemorrhagic shock.

The data sets for the concentration of each marker were skewed therefore the medians were compared. Significant differences were seen between all 3 conditions (in all pairwise combinations) in all markers except hCGh, hCGt×hCGh×hCGO×progesterone×CA125 (THBPC), and THB for ectopic pregnancy against spontaneous miscarriage; and CA125 and hCGt/hCGh ratio (T/H) for viable pregnancy against spontaneous miscarriage (Table 2). All markers had significantly lower concentrations in ectopic pregnancy compared to viable pregnancy (Table 2 and FIG. 1). ROC analyses (FIG. 2 and Table 3) for ectopic pregnancy against viable pregnancy revealed that the best performing marker was hCGt×CA125 (TC) with area under curve (AUC) of 0.912. The best performing single marker was hCGt with AUC of 0.878. At a cutoff of −3736 mIU/mL, hCGt had 100% sensitivity, 76% specificity, 39% PPV, and 100% NPV (Table 3). In the category of ectopic pregnancy against spontaneous miscarriage, CA125 and TC were the best markers with AUC of 0.802 and 0.794, respectively (FIG. 2). At a cutoff of −42.029 U/mL, CA125 had 100% sensitivity, 43% specificity, 21% PPV, and 100% NPV; and at a cutoff of 43,276.8, TC had 92% sensitivity, 60% specificity, 26% PPV, and 98% NPV (Table 3). The overall performance of progesterone was poor compared to other markers (Table 3 and FIG. 2).

In an algorithm in which low level total hCG (<3736 mIU/mL) was used to detect all ectopic hCG (100% sensitivity) but had high false positive due to simultaneous detection of many pregnancies destined to miscarry, miscarriage (Table 4) resulted in a 40.9% false positive rate (defined as any non-ectopic pregnancy). The addition of low CA-125, at a cutoff of 41.98 U/mL, excluded many of the miscarrying intrauterine pregnancies; maintaining a 100% sensitivity and reducing the false positive rate to 24.9%. Although retrospectively, analysis of the ultrasound reports for evidence of a uterine pregnancy indicated that if ultrasound assessment had been added as a third stage test identified, a further 43 false positives (all subsequently identified as miscarriages) would have been excluded. Ultrasound at this stage would maintain a 100% sensitivity for ectopic pregnancy but reduce the false positive rate by a further 12% to 14.4% (see Table 4).

Discussion

The use of a single point predictor biomarker to identify early ectopic pregnancy would aid diagnosis at initial presentation, thereby facilitating medical intervention prior to life-threatening tubal rupture, and thus help reduce maternal mortality. To date, no such single predictor has been found. Clinicians rely on transvaginal ultrasound, which is sensitive if an adequate serum hCG assay is used and levels fall above the discriminatory zone (1000-1500 mIU/mL) and is also dependent on serial 48 hour measurements of hCG to determine the doubling time. However, the discriminatory zone approach is clearly flawed in detecting ectopic pregnancies below the threshold and would thus have missed more than half (15/27 or 56%) of the ectopic pregnancies in our study. The doubling time approach is also hindered by the diagnostic delay time of 1.5 days, and the potential tubal rupture which may occur outside the healthcare setting if the repeat hCG measurement is performed on an out-patient basis. A highly sensitive single-point biomarker algorithm would improve maternal safety by detecting all ectopic pregnancies at presentation, and desirably also possess enough specificity to reduce medical costs from the needless follow up of patients with viable pregnancy. The reduced diagnostic turnaround time would also help alleviate the anxiety of patients. Previously, in a smaller study, it has been found that single point measurements of serum hCG isoforms, particularly hCGβ and hCGt, had better predictive function than corresponding urinary isoforms in distinguishing women with ectopic pregnancy from women with viable pregnancy. However neither marker could distinguish ectopic pregnancy from spontaneous miscarriage. This much larger study, examined similar markers with further inclusion of hCGh, progesterone and CA125. These were in either single or combination algorithms (Table 1).

We found that all the markers had significantly lower concentrations (P<0.0001) in ectopic pregnancy compared to viable pregnancy, though performances were relatively less so with progesterone (P=0.0004), and hCGt/hCGh ratio (P=0.02) (Table 2). All markers, therefore, appeared to have the potential for discriminating between ectopic and viable pregnancy, thus corroborating the previous study. However, some markers appeared to be better than others, as indicated by ROC analysis (FIG. 2 and Table 3). The best performing marker was hCGt×CA125 (TC) with area under curve (AUC) of 0.912. At a cutoff of ≦80,893.5, serum TC measurement was able to detect women with ectopic pregnancy and exclude those with viable pregnancy with a sensitivity of 100%, specificity of 78%, positive predictive value (PPV) of 40%, and negative predictive value (NPV) of 100%; combining all biomarkers did not improve on this algorithm (FIG. 2). The best performing single marker, and thus potentially the most cost effective marker of choice, was hCGt with AUC of 0.878. At a cutoff of ≦3736 mIU/mL, hCGt had 100% sensitivity, 76% specificity, 39% PPV, and 100% NPV. At a cutoff of ≦7.5 ng/mL, hCGβ had 96% sensitivity, 74% specificity, 36% PPV, and 99% NPV (Table 3).

Progesterone was significantly different between all pairwise combinations of ectopic pregnancy, viable pregnancy and spontaneous miscarriage (Table 2). However, here serum progesterone levels in ectopic pregnancy were marginally higher than in spontaneous miscarriage (FIG. 1); At a similar cutoff of 20.4 ng/mL (Table 3), we found a sensitivity of only 65% for ectopic pregnancy. Overall, despite the significant all-pairwise Kruskal-Wallis results (Table 2), ROC analyses revealed that the performance of progesterone was poor, compared to the other markers (FIG. 2 and Table 3). This may be attributable to the wide range of overlap for serum progesterone between ectopic pregnancy, spontaneous miscarriage and viable pregnancy (FIG. 1) which was reflected in the wide confidence intervals (Table 3). We found that all the markers except THBPC, THB, and hCGh were discriminatory between ectopic pregnancy and spontaneous miscarriage (Table 2). This included hCGt (P=0.002) and hCGβ (P=0.014), ROC analyses indicated that CA125 and hCGT with CA-125 were the best markers to distinguish ectopic pregnancy from spontaneous miscarriage, with AUC of 0.802 and 0.794, respectively (FIG. 2). At a cutoff of ≦42.029 U/mL, CA125 had 100% sensitivity, 43% specificity, 21% PPV, and 100% NPV; and at a cutoff of ≦43,276.8, TC had 92% sensitivity, 60% specificity, 26% PPV, and 98% NPV (Table 3). CA125 appeared to distinguish ectopic pregnancy, from intrauterine pregnancy (spontaneous miscarriage and viable pregnancy) as a combined group (Table 2, and FIG. 1). This was consistent with previous studies where serum CA125 was found to be lower in ectopic pregnancy compared to viable pregnancy, but not between viable pregnancy and spontaneous miscarriage. The trend with hCGh (P=0.69) was consistent with previous work and appeared overall to discriminate viable pregnancy from non-viable pregnancy (ectopic pregnancy and miscarriage) as a group (Table 2). The non-discrimination of ectopic pregnancy from spontaneous miscarriage found in THBPC and THB may thus have been due to hCGh, which was the marker common to both.

For the construction of a diagnostic algorithm, we found that either the single marker hCGt, or the combination marker hCGt and CA125 (depending on cost implications), appeared to be most favorable in excluding viable pregnancy from ectopic pregnancy (Table 3). Similarly, CA125, or hCGt×CA125 (TC), appeared most favorable in excluding spontaneous miscarriage from ectopic pregnancy (Table 3), and would potentially further reduce the false positives obtained after excluding viable pregnancy. We thus simulated a combination algorithm for diagnosis of ectopic pregnancy (Table 4), where, a positive hCGt assay (i.e. <3737 mIU/mL) was followed by a CA125 assay (deemed positive when <41.98 U/mL). The combined algorithm produced 100% sensitivity (26/26), 75.1% specificity and 24.9% false positive rate (FPR) for identifying ectopic pregnancy, even at initial presentation. This is in comparison to the current protocol, where follow up was required to identify 57.7% (15/26) of ectopic pregnancies (Table 4). Furthermore, the algorithm would be safe in excluding 75.1% (263/350) of non-ectopic pregnancies compared with 64.9% (227/350) at initial presentation in the current protocol. Significantly, this indicates that 36 additional patients would have been spared the anxiety of a possible ectopic pregnancy representing 10% of our study population. The CA125 assay was useful in reducing the FPR from 40.9% to 24.9% without compromising the 100% sensitivity (Table 4). Since 43 of the residual false positives had a gestation of >5.5 weeks, the FPR could potentially be reduced further to 14.37% with the application of ultrasonography, based on the detection capability for localising pregnancies with gestation of >5.5 weeks (at which stage the yolk sac is visible).

In conclusion, in early pregnancy, single-point serum measurements of hCGt <3737 mIU/mL together with CA125 <41.98 U/mL, are a promising algorithm for detecting ectopic pregnancy with 100% sensitivity, 75.1% specificity, and 24.9% false positive rate. The addendum of ultrasonography to the algorithm has potential to further reduce the false positive rate significantly, and needs to be explored. This algorithm has the advantage of a single point detection over current protocols which require follow up, and appears safe in excluding non-ectopic pregnancy at presentation. Indeed, the adoption of this biochemical screening would have detected the two women (8%) who were not identified as ectopic pregnancy by EPAU at presentation or follow up and subsequently presented with hemorrhagic shock, however a significant number of spontaneous miscarriage are also detected. Nevertheless this biochemical algorithm enables the identification of women with a high risk of ectopic pregnancy in early pregnancy and allows or intervention antecedent to tubal rupture and a reduction in maternal morbidity.

TABLE 1
Descriptive staustics for concentrations of hCGt, hCGB, hCGh, progesterone and CA125
in the sera of women with viable pregnancy, spontaneous miscarriage, and ectopic pregnancy.

| | Statistics | Viable pregnancy | | Spontaneous miscarriage | | Ectopic pregnancy | |
|---|---|---|---|---|---|---|---|
| Gestation, weeks | Mean (SD) | 7.3 | (2.4) | 7.6 | (2.3) | 6.3 | (2.9) |
| | Range | 1-12 | | 2-12 | | 0.7-12 | |
| hCGt, mlU/ml. | Mean (SD) | 47,490 | (52,525) | 11,702 | (25,840) | 1152 | (1048) |
| | Median (IQR) | 27,644 | (3768-81,237) | 2546 | (588-10,957) | 781 | (364-1806) |
| hCGβ, ng/mL | Mean (SD) | 24 | (20) | 8 | (11) | 3 | (3) |
| | Median (IQR) | 22 | (6-38) | 4 | (1-11) | 2 | (1-4) |
| hCGh, ng/mL | Mean (SD) | 794 | (1729) | 154 | (369) | 31 (30)31 (30) | |
| | Median (IQR) | 439 | (118-908) | 17 | (6-58) | 22 | (10-45) |
| Progesterone, ng/mL | Mean (SD) | 48 | (19) | 21 | (22) | 31 | (22) |
| | Median (IQR) | 56 | (35-64) | 15 | (5-29) | 25 | (13-55) |
| CA125, U/mL | Mean (SD) | 49 | (66) | 69 | (99) | 15 | (12) |
| | Median (IQR) | 27 | (15-21) | 31 | (16-70) | 10 | (6-22) |
| T^{a} × B^{b} | Mean (SD) | 1,855,125 | (2,584,570) | 309,408 | (1,100,852) | 5093 | (8588) |
| | Median (IQR) | 578,909 | (19,250-2,690,350) | 8789 | (782-91,162) | 1851 | (196-6892) |
| T^{a} × B^{b} × C [symbol] | Mean (SD) | 107,551,463 | (314,125,661) | 20,371,636 | (87,192,559) | 70,312 | (106,167) |
| | Median (IQR) | 16,132,936 | (615,576-93,587,040) | 317,905 | (29,178-4,438,013) | 10,676 | (4221-100,402) |
| T^{a}/H^{d} | Mean (SD) | 314 | (1190) | 233 | (612) | 59 | (58) |
| | Median (IQR) | 71 | (37-138) | 96 | (44-146) | 38 | (27-75) |
| Log [symbol] (T^{a} × H [symbol] × B^{b}) | Mean (SD) | 8 | (2) | 5 | (2) | 4 | (1) |
| | Median (IQR) | 9 | (7-9) | 5 | (4-6) | 5 | (4-5) |
| Log₁₀ (T^{a} × H^{d} × B^{b} × | Mean (SD) | 11 | (2) | 7 | (3) | 7 | (1) |
| P [symbol] × C [symbol] ) | Median (IQR) | 12 | (10-13) | 7 | (5-9) | 7 | (6-8) |
| T^{a} x C [symbol] | Mean (SD) | 2,637,744 | (6,480,122) | 775,330 | (2,211,175) | 18,153 | (22,418) |
| | Median (IQR) | 662,847 | (99,111-2,660,938) | 90,293 | (16,558-441,047) | 7005 | (3530-29,825) |

^{a}hCGt.
^{b}hCGβ.
^{c}CA125.
^{d}hCGh.
^{e}Progesterone. [symbol]
indicates data missing or illegible when filed

TABLE 2
Kruskal-Wallis all pairwise comparisons (Conover-
inman) of hCGt, hCGβ, hCGh, progesterone and CA125
in the sera of women with viable pregnancym spontaneous
miscarriage, and ectopic pregnancy at presentation.

| Event | Marker | P-value | Result |
|---|---|---|---|
| Ep vs VP | T | P < 0.0001 | Significant |
| | TBC | P < 0.0001 | Significant |
| | TB | P < 0.0001 | Significant |
| | THBPC | P < 0.0001 | Significant |
| | THB | P < 0.0001 | Significant |
| | H | P < 0.0001 | Significant |
| | B | P < 0.0001 | Significant |
| | C | P < 0.0001 | Significant |
| | TC | P < 0.0001 | Significant |
| | P | P = 0.0004 | Significant |
| | T/H | P = 0.0228 | Significant |
| EP vs. MIS | TC | P < 0.0001 | Significant |
| | C | P < 0.0001 | Significant |
| | TBC | P = 0.0001 | Significant |
| | T | P = 0.0018 | Significant |
| | TB | P = 0.0033 | Significant |
| | T/H | P = 0.004 | Significant |
| | P | P = 0.0046 | Significant |
| | B | P = 0.0135 | Significant |
| | THB | P = 0.2717 | Not significant |
| | THBPC | P = 0.468 | Not significant |
| | H | P = 0.687 | Not significant |
| VP vs. MIS | T | P < 0.0001 | Significant |
| | TC | P < 0.0001 | Significant |
| | TBC | P < 0.0001 | Significant |
| | P | P < 0.0001 | Significant |
| | THBPC | P < 0.0001 | Significant |
| | THB | P < 0.0001 | Significant |
| | H | P < 0.0001 | Significant |
| | B | P < 0.0001 | Significant |
| | C | P = 0.0714 | Not significant |
| | T/H | P = 0.25 | Not significant |

T. hCGt;
B, hCGβ;
H, hCGh;
C, CA125:
P, progesterone.

TABLE 3
Ranked comparison of ROC performances of serum hCGt, hCGβ, hCGh, progesterone and
CA125 for (top), the inclusion of ectopic pregnancy (EP) and exclusion of viable pregnancy
(VP); and (bottom), inclusion of EP with exclusion of spontaneous miscarriage (MIS).

| | | | | | | | | |
|---|---|---|---|---|---|---|---|---|
| Marker | Sensitivity | 95% CI | specificity | 95% CI | PPV | NPV | Cutoff≦ | Area under curve |

EP vs. VP

| TC | 1.00 | 0.87-1.00 | 0.78 | 0.71-84 | 0.40 | 1.00 | 80,893.5 | | 0.912 |
|---|---|---|---|---|---|---|---|---|---|
| TBC | 1.00 | 0.87-1.00 | 0.77 | 0.70-0.83 | 0.39 | 1.00 | 411,745 | | 0.901 |
| THB | 1.00 | 0.83-1.00 | 0.77 | 0.67-0.86 | 0.51 | 1.00 | 6.33 | | 0.895 |
| THBPC | 1.00 | 0.87-1.00 | 0.79 | 0.69-0.88 | 0.54 | 1.00 | 9.33 | | 0.892 |
| T | 1.00 | 0.87-1.00 | 0.76 | 0.69-0.82 | 0.39 | 1.00 | 3736 | mIU/mL | 0.878 |
| TB | 1.00 | 0.87-1.00 | 0.74 | 0.67-0.80 | 0.37 | 1.00 | 38,480 | | 0.877 |
| H | 1.00 | 0.83-100 | 0.76 | 0.66-0.85 | 0.50 | 1.00 | 112.2 | ng/mL | 0.869 |
| B | 0.96 | 0.83-100 | 0.74 | 0.61-0.81 | 0.36 | 0.99 | 75 | ng/mL | 0.857 |
| C | 0.58 | 0.32-0.77 | 0.85 | 0.81-0.95 | 0.36 | 0.93 | 11.4 | U/mL | 0.775 |
| P | 0.73 | 0.52-0.88 | 0.70 | 0.62-0.76 | 0.26 | 0.95 | 44 | ng/mL | 0.704 |

| | | | | | | | | |
|---|---|---|---|---|---|---|---|---|
| T/H | 0.70 | 0.46-0.88 | 0.61 | 0.51-0.72 | 0.30 | 0.90 | 47.3 | 0.660 |

EP vs. MIS

| C | 1.00 | 0.87-1.00 | 0.43 | 0.36-0.51 | 0.21 | 1.00 | 41.98 | U/mL | 0.802 |
|---|---|---|---|---|---|---|---|---|---|
| TC | 0.92 | 0.75-0.99 | 0.60 | 0.53-0.68 | 0.26 | 0.98 | 43,276.8 | | 0.794 |
| TCB | 0.96 | 0.80-.99 | 0.51 | 0.43-0.59 | 0.23 | 0.99 | 301,999 | | 0.744 |
| T/H | 0.75 | 0.51-0.91 | 0.67 | 0.51-0.79 | 0.50 | 0.86 | 61.8 | | 0.730 |
| T | 0.89 | 0.71-0.98 | 0.54 | 0.46-0.62 | 0.23 | 0.97 | 2076 | mIU/mL | 0.694 |
| TB | 0.96 | 0.81-0.99 | 0.42 | 0.35-0.49 | 0.21 | 0.99 | 23,378 | | 0.684 |
| P | 0.65 | 0.44-0.83 | 0.63 | 0.56-0.71 | 0.21 | 0.92 | 20.4 | ng/mL^{a} | 0.667 |
| B | 0.85 | 0.66-0.96 | 0.47 | 0.40-0.55 | 0.20 | 0.95 | 3.96 | ng/mL | 0.639 |
| THB | 1.00 | 0.83-.100 | 0.24 | 0.13-0.39 | 0.37 | 1.00 | 6.33 | | 0.572 |
| THBPC | 0.95 | 0.75-.099 | 0.35 | 0.21-0.51 | 0.40 | 0.94 | 8.44 | | 0.536 |
| H | 0.95 | 0.83-1.00 | 0.27 | 0.09-0.34 | 0.37 | 0.92 | 5.9 | ng/mL | 0.523 |

T, hCGt;
B, hCGβ;
H, hCGh;
C, CA125;
P, progesterone
^{a}≧

TABLE 4
Diagnostic representation of the step wise application of a biochemical algorithm for ectopic pregnancy coupled with ultrasound scanning.

| Current algorithm performance | Viable pregnancy | Miscarriage | Ectopic pregnancy | Total |
|---|---|---|---|---|
| Diagnosed at presentation | 122 (69.7%) | 105 (60%) | 11 (42.3%) | 238 |
| Diagnosed at follow up | 53 (30.3%) | 70 (40%) | 15 (57.7%) | 138 |
| Total | 175 | 175 | 26 | 376 |
| Suggested 3 stage analysis | | | | |
| | 1st stage analysis of serum samples taken at presentation | | | |
| | Negative result | | | |
| A. hCGt assay <3736 mlU/mL | (non-ectopic pregnancy) | | Positive result | Total |
| Outcome interpretation | True negative | False positive | Sensitivity—true positive | |
| Performance | 207 (59.1%) | 143 (40.9%) | 26 (100%) | 376 |
| | 2nd stage analysis of samples scored positive in A | | | |
| B. CA125 assay <41.98 U/mL | 56* (16%) | 87 (24.9%) | 26 (100%) | 169 |
| Performance (combined effect of stages A and B | 2643 (75.1%) | 87 (24.9%) | 26 (100%) | 376 |
| immunoassay of serum samples taken at presentation) | | | | |
| | 3rd stage ultrasound of patients scored psoitive in B | | | |
| Potential of an additional third stage | True negative | False positive | Sensitivity—true positive | |
| Based on 43/87 false positives identified by serum | 306 (87.4%) | 44 (14.4%) | 26 (100%) | 376 |
| immunoassay (gestational age >5.5 weeks) reclassified | | | | |
| as non-ectopic following ultrasound scan. | | | | |

1. A method of determining the likelihood of an ectopic pregnancy or spontaneous miscarriage comprising measuring the level of one or more markers selected from total hCG(hCGt), free beta subunit of hCG (hCGβ), hyperglycosylated form of hCG (hCGh), CA125 and progesterone in a sample obtained from a pregnant woman.

2. The method of claim 1 wherein the levels of combination of two or more of said markers is measured.

3. The method of claim 1 wherein an ectopic pregnancy can be distinguished from a normal viable intrauterine pregnancy.

4. The method of claim 1 wherein the level of hCGt is measured.

5. The method of claim 1 wherein the levels of hCGt and CA125 are measured.

6. The method of claim 1 wherein the level of hCGh is measured.

7. The method of claim 1 further comprising analysing the results obtained from an ultrasound scan to determine the presence of a foetal sac and optionally a foetal heartbeat.

8. The method of claim 1 wherein a woman likely to suffer a spontaneous miscarriage can be distinguished from a normal viable intrauterine pregnancy.

9. The method of claim 1 wherein a woman likely to suffer a spontaneous miscarriage can be distinguished from an ectopic pregnancy.

10. A method according to claim 1, wherein the sample is from a pregnant woman at between 4 and 10 weeks gestation.

11. The method of claim 1 wherein the sample is a serum sample.
